(19) United States
(12) Patent Application Publication
(10) Pub. No.: US 2011/0124607 A1
(43) Pub. Date: May 26, 2011

(54) COMPOSITION FOR REGULATION CELLULAR SENESCENCE COMPRISING LYSOPHOSPHATIDIC ACID AND INHIBITOR OF ADENYLYL CYCLASE AS ACTIVE INGREDIENTS

(76) Inventors: Sang Chul Park, Gyeonggi-do (KR); Eui Ju Yeo, Gyeonggi-do (KR); Ji Heon Rhim, Seoul (KR)

(21) Appl. No.: 12/736,768

(22) PCT Filed: May 14, 2008
(86) PCT No.: PCT/KR2008/002685

§ 371 Date: Dec. 15, 2010

(51) Int. Cl. A61K 31/661; C12N 5/071; A61P 43/00

(52) U.S. Cl. 514/120; 435/375

(57) ABSTRACT

The present invention relates to the molecular mechanism inducing cell proliferation in aged human fibroblasts by inhibiting AMPK using LPA and AC inhibitor. Particularly, the present invention relates to a composition comprising LPA and ACI as active ingredients and the invention proves with the said composition that LPA and ACI regulate different phosphorylation of AMPKα and thus inactivate p53 and induce senescent cell proliferation. This results support the fact that AMPK signal transduction plays an important role in cell proliferation of senescent cells.

### TECHNICAL FIELD

The present invention relates to a composition for regulating cellular senescence comprising lysophosphatidic acid (LPA) and adenylyl cyclase (ACI) as active ingredients, more precisely a composition for regulating cellular senescence comprising LPA and ACI as active ingredients and a method for regulating cellular senescence containing the step of treating effective dose of the said composition to senescent cells.

### BACKGROUND ART

Cellular senescence plays an important role in complicated biological processes including development, maturity and tumorigenesis. So, numbers of attempts have been made to understand basic but important characteristics of cellular senescence (Peacocke and Campisi, 1991; Smith and Pereira-Smith, 1996). One of the characteristics of cellular senescence is hyporesponsiveness to growth factor and mitogen.

Lysophosphatidic acid (LPA) is an important mitogen agonist which induces signal transduction in relation to intracellular Ca²⁺ transport, actin polymerization and phosphatidic acid generation in human bigerminal fibroblasts, and acts as an extracellular messenger through guanine nucleotide binding protein (G-protein). LPA is also known as the material having various biological effects on cell morphology, chemotaxis and differentiation mediated by LPA receptor (Moolenaar, 2000; Moolenaar et al., 1997). LPA receptor is exemplified by such isotypes as LPA1, LPA2 and LPA3 and these isotypes are bound to Giα which is sensitive to pertussis toxin to inhibit adenylyl cyclase activity (An et al., 1998), resulting in the decrease of cAMP (Taussig et al., 1993).

Interestingly, LPA reduces cAMP in young cells but increases cAMP in senescent cells, indicating that it regulates lower signal transduction system (Jang et al., 2006a; Jang et al., 2003; Jang et al., 2006b). The interaction between cAMP signal transduction and AMPK signal transduction is well known in muscle, liver and adipocyte (Cohen and Hardie, 1991; Kahn et al., 2005; Long and Zierath, 2006). Mammalian AMPK is a protein having serine/threonine kinase activity, which is composed of catalytic subunit α and two regulatory subunits β and γ. AMPK is activated when Thr172 located in activating loop of α subunit is phosphorylated. When AMP, the most important factor for regulating AMPK activity, is bound to γ subunit, phosphorylation mediated by upstream kinase of AMPK (known as AMPKK) is induced. AMPKK is exemplified by LKB1/STK11 which was identified as mutated in Peutz-Jeghers syndrome (Hawley et al., 2003; Shaw et al., 2004; Woods et al., 2003a), calcium/calmodulin dependent protein kinase kinase (CaMKK)-α and β (Hawley et al, 2005; Hong et al., 2005; Hurley et al., 2005; Woods et al., 2005), and TAK1 (Woods et al., 2003a).

There are other phosphorylation sites of AMPK identified in α and β subunits in addition to Thr172. However, it has not been confirmed yet whether these sites are involved in the regulation of AMPK activity (Mitchelhill et al., 1997; Stein et al., 2000; Warden et al., 2001; Woods et al., 2003b). In particular, Ser485 of AMPKα1 (corresponding to Ser491 of AMPKα2) is auto-phosphorylation site (Horman et al., 2006) which is phosphorylated by PKA (Hurley et al., 2006) or protein kinase B(PKB)/AKT (Hahn-Windgassen et al., 2005; Horman et al., 2006; Soltys et al., 2006). Phosphorylation of Ser485/491 by PKA or PKB/AKT inhibits approach of α-Thr-172, resulting in the decrease of Thr-172 phosphorylation. As a result, AMPK activation is inhibited.

Tumor suppressor gene product p53 is activated by AMPK mediated phosphorylation of Ser 15. This process is essential for the protein to migrate into nucleus and have transcription activity. Transcription activity of p53 is involved in the regulation of the level of p21 protein acting as p53-dependent cyclin-dependent kinase (cdk) inhibitor. Cdk is an important enzyme controlling cell cycle of a eukaryotic cell. When a normal eukaryotic cell receives growth signal via signal transduction pathway, cell proliferation is induced according to a series of cell cycle. At this time, cdk is conjugated to cyclin specifically expressed in each stage of cell cycle to form a functional unit, thereby specific cyclin-cdk complex which activates each stage of cell cycle is formed. The activation of cyclin-cdk complex is regulated by various mechanisms. For example, cdk is phosphorylated or dephosphorylated or bound to a specific inhibitor protein, or cyclin might be proteolyzed. Cell cycle is regulated to be happening at a right time at a right place. Accurate regulation of cell cycle is controlled by various regulation factors including cyclin-cdk complex. P21 protein is an example of such regulation factors. Once DNA is damaged, tumor suppressor gene p53 is activated and thus activated p53 induces p21 expression. P21 is bound to cyclin-cdk complex inducing S-phase, leading to the inhibition of CDK 4/6/2 kinase activity. As a result, phosphorylation of Rb is inhibited. Then, cells are arrested in G1 stage to earn time for DNA repair.

AMPK is known to induce p53 phosphorylation and thereby increase p21 expression, resulting in the inhibition of cell proliferation. However, various theories on cell proliferation of intracellular molecular species are proposed, so more clear explanation on such phenomena is required.

### DISCLOSURE

### Technical Problem

The present inventors tried to disclose more details of intracellular molecular species and signal transduction system involved in cell proliferation. As a result, the inventors found out that LPA induces cell proliferation in both young cells and senescent cells, while ACI reduces cell proliferation in young cells but induces cell proliferation in senescent cells. And the inventors further confirmed that AMPK is deeply involved in such processes. In conclusion, the present inventors proved that LPA and ACI regulate AMPK phosphorylation differently to reduce AMPK activation and as a result senescent cells are proliferated. And the inventors further confirmed that co-treatment of LPA and ACI induced cell proliferation more effectively than single treatment of LPA or ACI.

### TECHNICAL SOLUTION

It is an object of the present invention to provide a composition for regulating cellular senescence comprising LPA and ACI as active ingredients.

It is another object of the present invention to provide a method for regulating cellular senescence containing the step of treating effective dose of LPA and ACI to senescent cells.

It is also an object of the present invention to provide a method for regulating cellular senescence containing the step of administering the composition comprising LPA and ACI to a subject in need of regulating cellular senescence.

Other objects and advantages of the present invention are disclosed by the appended claims and the following embodiments including figures.

### Advantageous Effects

The present invention relates to a composition for regulating cellular senescence comprising LPA and ACI as active ingredients and a method for regulating cellular senescence containing the step of treating effective dose of the said composition to senescent cells. The composition for regulating cellular senescence of the present invention and the method for regulating cellular senescence using the same are effective in controlling cellular senescence of senescent cells.

### DESCRIPTION OF DRAWINGS

The above and other objects, features and advantages of the present invention will become apparent from the following description of preferred embodiments given in conjunction with the accompanying drawings, in which:

FIG. 1 is a set of graphs illustrating the effect of LPA and ACI on cell proliferation and entry to S phase of senescent cells. (A) and (B) are graphs illustrating the results of counting cells after 1, 2 and 4 day of culture after treating sub-cultured young cells (PD 20: A) and aged cells (PD 64: B) with LPA and ACI singly or together. (C) is a graph illustrating the result of counting young and senescent cells which were serum-starved for 2 days to synchronize cells at the G0/G1 phase and then treated with LPA and ACI singly or together, followed by culture for 1, 2 and 4 days.

At this time, cell numbers regarded as significant compared with that of the control were presented in (A) and (B) (p<0.001). Cell cycle in (C) was analysed by flow cytometry. The percentage of cells entered S phase was averaged of three repeated measurements (p<0.001).

FIG. 2 is a graph illustrating the result of soft agar assay saying that LPA and ACI did not form colony in both young and senescent cells. Young and senescent cells were distributed in DMEM containing 10% bovine serum and 0.3% top agar, which were loaded on 0.6% basic agar layer in 60 mm culture dish. The cells treated with 30 μM LPA (L), 300 μM ACI (A), or both LPA and ACI for three weeks were fixed with 70% ethanol, and so was the control treated with nothing. After staining the cells with trypan blue, colonies were counted under microscope. HeLa and HepG2 cancer cell lines were distributed on soft agar dish, as positive controls, followed by treatment with LPA, ACI, or LPA+ACI. Colony formation was also analysed. The number of colonies formed in soft agar dish was plotted as mean+/− standard deviation and each measurement was repeated at least three times.

FIG. 3 is a set of photographs illustrating the effect of LPA and ACI on the expressions of p21 and cyclin-D1 in young and senescent cells. Particularly, a-f, g-l and m-r are photographs illustrating that sub-cultured young cells (PD 20: Y) and senescent cells (PD 65: S) were serum-starved for 48 hours respectively, followed by treatment of LPA (a-f) and ACI (g-l) singly or together (m-r), and after culturing for 1, 2, and 4 days, the cells were fixed in 4% hydrogen peroxide, and stained with p21waf1/cip1 (A) and cyclin D1 (B) antibodies, followed by confirming immunofluorescence. At this time, nucleus was stained with DAPI.

FIG. 4 is a set of photographs illustrating the results of investigation of AMPK expression level in young and senescent cells, and back skins of young and aged men.

(A) is a set of photographs illustrating the results of Western blotting examining the expression level of AMPKα, p-Thr172-AMPKα, p-Ser485/491-AMPKα, p53, p-Ser15-p53, p21waf1/cip1 and β-actin using 45 μg of the protein extracted from sub-cultured young cells (PD 20: Y) and aged cells (PD 64: S).

(B) is a set of photographs illustrating the expression level of proteins in sub-cultured young cells (a, c, e, g, i) and senescent cells (b, d, f, h, j) fixed and stained with anti-AMPKα (a, b), anti-p-Thr172-AMPKα (c, d), anti-anti-p-Ser485/491-AMPKα (e, f), anti-p53 (g, h), and anti-p-Ser15-p53 (i, j). At this time, nucleus was stained with DAPI.

(C) is a set of photographs illustrating the expression level of AMPKα (a, b), p-Thr172-AMPKα (c, d), p53 (e, f), and anti-p-Ser15-p53 (g, h) in back skins of a 10 year old boy (a, c, e, g) and a 58 year old man (b, d, f, h) were detected by immunohistochemistry as described in Materials and Methods. Each experiment was repeated three times and the same results were obtained.

FIG. 5 is a set of photographs and graphs illustrating the effects of AICAR and AMPKI on the activation of AMPK and cell proliferation in young and senescent cells.

(A) and (B): Young and senescent cells were serum-starved for 2 days, followed by treatment with 10 mM of AMPK inhibitor AMPKI(A) or 10 mM of AMPK activator AICAR(B) for 4 days. Proteins were extracted from the treated cells and the levels of AMPKα and p-Thr172-AMPKα, total p53, p-Ser15-p53 and p21waf1/cip1 were measured by immuno-blotting. (C) and (D): Young cells (C) and senescent cells (D) were treated with 10 mM AMPKI or 10 mM AICAR and cultured for 4 days, followed by measurement of cell proliferation by cell counting. At this time, the experiment was repeated three times (p<0.001).

FIG. 6 is a set of photographs illustrating the effects of LPA and ACI on AMPK and p53 phosphorylation in young and senescent cells.

(A) and (B) are photographs illustrating the results of immuno-blotting. Precisely, sub-cultured young cells (PD 18: A) and senescent cells (PD 64: B) were treated with 30 μM of LPA or 300 μM of LPA and ACI singly or together, followed by further culture for 1, 2, and 4 days. Proteins were extracted from the cultured cells and the levels of AMPKα, p-Thr172-AMPKα, p-Ser485/491-AMPKα, p53, p-Ser15-p53, p21waf1/cip1 and β-actin were quantified by immuno-blotting.

FIG. 7 is a set of photographs illustrating the effects of LPA and ACI on AMPK phosphorylation in senescent cells treated with PKA inhibitor Rp-cAMP.

(A) and (B) are photographs illustrating the results of immuno-blotting. Precisely, senescent cells (PD 64) were pre-treated with 10 mM of PKA inhibitor Rp-cAMP for one hour, followed by treatment with LPA(A) or ACI(B). After culturing the cells for 1, 2, and 4 days, proteins were extracted from the cultured cells and the levels of AMPKα, p-Thr172-AMPKα, p-Ser485/491-AMPKα, p53, p-Ser15-p53, p21waf1/cip1 and β-actin were quantified by immuno-blotting using 45 μg of the protein.

FIG. 8 is a set of photographs illustrating the effects of LPA and ACI on LKB1 phosphorylation in senescent cells. Precisely, sub-cultured young cells (PD 18) and senescent cells (PD64) were treated with 30 μM of LPA or 300 μM of LPA and ACI singly or together. After culturing the cells for 1, 2, and 4 days, proteins were extracted from the cultured cells and the levels of LKB1, p-Ser431-LKB1 and β-actin were quantified by immuno-blotting using 45 μg of the protein.

FIG. 9 is a set of schematic diagrams illustrating the regulation of AMPK activity by LPA and ACI in senescent cells.

(A) is a schematic diagram illustrating the effect of LPA and ACI in young cells. When young cells were treated with LPA, cAMP was down-regulated and PKA activity was inhibited. As a result, p-Ser485/491-AMPK activity inducing AMPK activity was reduced, resulting in the decrease of AMP activity. LPA also reduced PKA dependent LKB1 phosphorylation. And also, it reduced p-Thr172-AMPK inactivating AMPK, resulting in the inhibition of AMPK activation. In the meantime, ACI reduced cAMP/PKA, and thus inhibited p-Ser485/491-AMPKα phosphorylation. It activated LKB1 a bit. As a result, p-Thr172-AMPKα phosphorylation was increased, resulting in the activation of AMPK. In young cells, cell proliferation was rather reduced by ACI.

(B) is a schematic diagram illustrating the effect of LPA and ACI in senescent cells. When senescent cells were treated with LPA, LPA increased cAMP level to activate PKA. As a result, AMPKα on Ser485/491 phosphorylation was increased to reduce AMPK activity and at the same time reduced p-Thr172-AMPKα phosphorylation to reduce AMPK activity. On the other hand, ACI did not alter in p-Ser485/491-AMPKα phosphorylation and only reduced LKB1 and LKB1 phosphorylation. As a result, ACI had the effect of reducing p-Thr172-AMPKα phosphorylation to decrease AMPK activity.

### BEST MODE

The present invention relates to a composition for regulating cellular senescence comprising LPA and ACI as active ingredients.

The present invention also relates to a method for regulating cellular senescence containing the step of treating effective dose of LPA and ACI to senescent cells.

The terms “senescence” used in this description has the same meaning as “aging”. In relation to cells, the term “young cell” indicates presenescent young cell. Unless stated otherwise, every technological and scientific terms used in this invention are understood as conventional meaning accepted by those in the art. For example, terms used in this description are all found in Benjamin Lewin, Genes VII (Oxford University Press (2000); and Kendrew et al., The Encyclopedia of Molecular Biology (Blackwell Science Ltd. (1994)).

In a preferred embodiment of the present invention, the cells appropriated for this invention are preferably derived from mammalian including human, pig, and cow, and particularly human cells are more preferred and specifically human fibroblasts are most preferred.

The method of the present invention can be applied to any senescent cells. But, considering treatment effect, important target cells are (a) those cells having replicative capacity in central nervous system, for example astrocytes, endothelial cells and fibroblasts playing an important role in aging-related disease such as Alzheimer's disease, Parkinson's disease, Huntington's disease and stroke; (b) those cells having limited replicative capacity in integument, for example fibroblasts, sebaceous cells, melanocytes, keratinocytes, Langerhans cells and follicle cells playing an important role in integument aging-related disease such as skin atropy, elastolysis, wrinkles, sebaceous hyperplasia, lentigo senile, hair whitening, hair loss, chronic cutaneous ulcer and aging-related wound healing capacity loss; (c) those cells having limited replicative capacity in articular cartilage, for example chondrocytes, lacunal and synovial fibroblasts playing an important role in degenerative joint disease; (d) those cells having limited replicative capacity in bone, for example osteoblast, stromal fibroblasts and osteoprogenitor cells playing an important role in osteoporosis; (e) those cells having limited replicative capacity in immune system, for example B and T lymphocytes, monocytes, neutrophils, eosinophils, basophilic leukocytes, NK cells and their precursor cells playing an important role in aging-related immune malfunction; (f) those cells having limited replicative capacity in vascular system, for example epidermal cells, smooth muscle cells and adventitial fibroblasts playing an important role in aging-related disease of vascular system such as arteriosclerosis, calcification, thrombus and aneurysm; and (g) those cells having limited replicative capacity in eye, for example pigmented epithelial cells and vascular endothelial cells playing an important role in macular degeneration.

In a preferred embodiment of the present invention, when LPA is treated alone to senescent cells, intracellular cAMP level is increased. In the meantime, when adenylyl cyclase (ACI) is treated alone, downstream signal transduction is completely blocked by PKA in young and senescent cells. ACI treatment results in the decrease of cell number in young cells but the increase of cell number in senescent cells. In addition, ACI suppresses p21 and cyclin D1 expressions in senescent cells to promote the entry to S phase and thus changes senescent cells to young cell-like cells. In the meantime, co-treatment of LPA and ACI brings greater effect on the promotion of cell proliferation than single treatment of LPA or ACI. This phenomenon is not consistent with that in young cells. When LPA and ACI are treated to senescent cells, intracellular AMPK activity is reduced, suggesting that LPA and ACI regulate AMPK activity separately and differently and are involved in Thr172-AMPKα phosphorylation differently. Thus, regulation of senescence by LPA and ACI is related to AMPK activity.

In the composition for regulating cellular senescence and the method for regulating cellular senescence of the present invention, LPA and ACI can be treated simultaneously or treated stepwise regardless of order. The effective doses of LPA and ACI for regulating cellular senescence is 1-50 μM and 1-500 μM respectively, and more preferably 30-50 μM and 200-300 μM.

The said adenylyl cyclase inhibitor is preferably selected from the group consisting of 2′,5′-dideoxyadenosine, cis-N-(2-phenylcyclopentyl)azacyclotridec-1-en-2-amine (MDL12,330A hydrochloride), and 9-(tetrahydro-2′-furyl)adenine (SQ22536), and more preferably 9-(tetrahydro-2′-furyl)adenine, but not always limited thereto.

The composition of the present invention can contain a proper amount of salt and a buffer containing pH regulator in order to maintain maximum physiological activity of the active ingredient. To be effective, the active ingredient of the present invention can be mixed with a dispersing agent or a stabilizer for administration.

When the composition of the present invention contains a protein, the composition can contain a pharmaceutically acceptable carrier which is exemplified by carbohydrate (ex: lactose, amylose, dextrose, sucrose, sorbitol, mannitol, starch, cellulose, etc), acacia rubber, calcium phosphate, alginate, gelatin, calcium silicate, microcrystalline cellulose, polyvinyl pyrrolidone, cellulose, water, syrup, salt solution, alcohol, Arabia rubber, vegetable oil (ex: corn oil, cotton seed oil, soybean oil, olive oil, coconut oil, etc), polyethylene glycol, methyl cellulose, methylhydroxy benzoate, propylhydroxy benzoate, talc, magnesium stearate and mineral oil, but not always limited thereto. The composition of the present invention can additionally contain lubricants, wetting agents, sweeteners, aromatics, emulsifiers, suspending agents, and preservatives, but not always limited thereto.

The composition of the present invention can be administered by any conventional pathway that is available for any pharmaceutically acceptable composition, particularly by transdermal, oral or parenteral administration. Parenteral administration is exemplified by intravenous injection, hypodermic injection, and intramuscular injection, and intramuscular injection is preferred.

The effective dose of the composition of the present invention can be administered by any method used for generally accepted pharmaceutical composition and the dose varies from formulation method, administration pathway, age, weight, gender, health condition, diet, administration frequency, administration method, excretion and sensitivity, and can be determined by an experienced doctor by considering the effectiveness in prevention or treatment.

The composition of the present invention can be formulated by the method that can be performed easily by those in the art by using a pharmaceutically acceptable carrier and/or excipient in the form of unit dose or in multi-dose container. The formulation can be in the form of solution, suspension or emulsion in oil or water-soluble medium, extract, powder, granule, tablet or capsule. At this time, a dispersing agent or a stabilizer can be additionally included. To maintain the maximum physiological activity of the active ingredient, a buffer containing proper amount of salt and pH regulator can be added.

The present invention also relates to a method for regulating cellular senescence containing the step of administering the composition of the present invention comprising LPA and ACI as active ingredients to a subject in need of regulating cellular senescence.

The method for regulating cellular senescence of the present invention is highly effective in the improvement and treatment of aging-related disease by administering the composition comprising LPA and ACI as active ingredients to a target subject. And, the composition comprising LPA and ACI and the method for regulating cellular senescence by treating the said composition to target cells are as described above.

The ‘aging-related disease’ herein is exemplified by central nervous system disease such as Alzheimer's disease, Parkinson's disease, Huntington's disease and stroke; integument disease such as skin atropy, elastolysis, wrinkles, sebaceous hyperplasia, lentigo senile, hair whitening, hair loss, chronic cutaneous ulcer and aging-related wound healing capacity loss; articular cartilage disease such as degenerative joint disease and osteoporosis; immune system disease; vascular system disease such arteriosclerosis, calcification, thrombus and aneurysm; and ophthalmic disease such as macular degeneration, but not always limited thereto.

The target subject of the present invention can be any mammals including human, and preferably human.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE

1. Materials

Dulbecco's modified Eagle's medium (DMEM: JBI) was used as the medium for cell culture herein and LPA, propidium iodide (PI) and trypan blue were purchased from Sigma (St. Louis, Mo., USA). 10% Fetal Bovine Serum (FBS), penicillin and streptomycin, the antibiotics used for cell culture, were purchased from Gibco/BRL (Carlsbad, Calif., USA). Polyclonal antibodies against AMPKα, p-Thr172-AMPKα, p-Ser485/491-AMPKα, p53, p-Ser15-p53 and p21WAF1/CIP1 were purchased from Cell Signaling (Beverly, Mass., USA). Polyclonal antibody against β-actin was purchased from Santa Cruz (Calif., USA). The PKA inhibitor Rp-cAMP and the AC inhibitor ACI (SQ22536) were purchased from CalBiochem (San Diego, Calif., USA). Horseradish peroxidase conjugated anti-rabbit-IgG and anti-mouse-IgG, the secondary antibodies, were purchased from Zymed (South San Francisco, Calif., USA). NC membrane (nitrocellulose membrane) for immuno-blotting was purchased from Schleicher& Schuell (Dassel, Germany). BCA (bicinchoninic acid) and ECL (enhanced chemiluminescence) set for protein quantification was purchased from Pierce-Biotechnology (Lockford, Ill., USA). Vectastain elite avidin-biotin complex kit for immunohistochemical staining was purchased from Vector laboratories (Burlingame, Calif., USA) and EnVision test system was purchased from DakoCytomation (Carpinteria, Calif., USA). Automation buffer was purchased from Biomeda (Foster City, Calif., USA).

2. Cell Culture

Human fibroblasts were prepared by primary culture of foreskin of a newborn baby (Boyce and Ham, 1983). The primary culture was performed in DMEM supplemented with 10% FBS and 1% antibiotics. The protein contents of young cells from the early stage of sub-culture, with a population doubling (PD) of less than 25, were compared to those of at least PD 65-70 senescent cells. Senescent cells were bigger in size than young cells and they showed morphological changes as being flat and multi-nuclei. In senescent cells, the activity of beta-galactosidase was increased and cell proliferation was reduced (Yeo et al., 2000).

Prior to LPA and ACI treatment, cells were grown for 1-2 days to 60-70% sub-confluence in DMEM-containing culture medium, and then serum-starved to quiescence (that is Go/G1 arrest) by incubation in a serum-free medium containing 0.1% bovine serum albumin (BSA) for 2 days. Young and senescent cells were treated with LPA, ACI, LPA+ACI, AMPKI and AICAR, respectively. Live cells were measured after staining the cells with trypan blue on day 1, day 2 and day 4 to confirm cell proliferation.

3. Protein Extraction and Immuno-Blotting

To analyse protein expression, human fibroblasts were lysed in cold lysis buffer (25 mM Hepes, pH 8.0, 150 mM NaCl, 1 mM EDTA, 1 mM Na₃VO₄, 1 mM NaF, 1% Triton X-100 and protease inhibitors), followed by centrifugation at 4° C. at 9,000 rpm for 10 minutes to obtain supernatant. Protein in the extract was quantified by BCL method. SDS-PAGE electrophoresis was performed with equal amount of the protein (45 μg) to separate. The separated protein was transferred onto NC membrane from the electrophoresis gel. Non-specific protein binding was blocked by blocking the blot (transferred NC membrane) using TTBS (Iris Buffered Saline with Tween-20) containing 5% skim milk for one hour. Then, antigen-antibody reaction was induced with diluted primary antibody at 4° C. for overnight. The blot was washed with TTBS, followed by reaction with horseradish peroxidase conjugated anti-IgGs diluted in TTBS containing 5% skim milk (1:5000) at room temperature for one hour. The blot was washed again with TTBS to eliminate non-specific binding of antigen-antibody. Photographs were developed/printed on X-ray film (Kodak) using ECL kit (Pierce) containing peroxidase substrate to identify each protein.

4. Immunofluorescent Staining

Coverslips were placed on 24 well plate on which required amounts of young and senescent cells were distributed. Media were eliminated and the treated cells were washed with PBS twice. Then, the cells were fixed with 4% hydrogen peroxide. Non-specific protein staining was blocked using PBS containing 2% BSA (blocking serum). The cells were stained with primary antibody such as anti-AMPKα, anti-p-Thr172-AMPKα, anti-p-Ser485/491-AMPKα, anti-p53, anti-p-Ser15-p53, anti-p21waf1/cip1, and anti-cyclin D1. To stain nucleus, DAPI (1:1000) was also added, followed by observation under Zeiss LSM 510 laser scanning microscope.

5. Immunohistochemical Staining

Human skin was biopsied and the obtained tissues were fixed in 4% (wt/vol) formalin dissolved in PBS (PH 7.4). The tissues were soaked in cold 10% hydrogen peroxide for overnight. Then, the tissues were embedded in paraffin and dissected to make 5-mm sections. Depraffination and hydration were performed with xylene and alcohol. The slides were boiled in 10 mM citrate buffer in microwave at 700 W for 10 minutes. The slides were soaked in 3% hydrogen peroxide for 15 minutes to block endogenous peroxidase action, followed by washing. The slides were reacted in 5% blocking serum for overnight to block non-specific protein staining. The slides were reacted with such primary antibodies as anti-AMPKα, p-Thr172-AMPKα, p53 and p-Ser15-p53 at room temperature for one hour at 1:100. The slides reacted with the primary antibody were washed three times, followed by reaction with secondary antibody at room temperature for 30 minutes. At this time, anti-rabbit antibody (DakoCytomation EnVision detection system) was used as the secondary antibody. After washing, the slides were reacted with HRP. After the reaction with HRP, the slides were stained with DAB. The slides were dehydrated with ethanol and then washed with xylene, followed by inclusion. The slides were photographed using Leica DEF 280 microscope (x200).

6. Cell Cycle Analysis

Young and senescent cells were treated with 30 μM of LPA, 300 μM of ACI, LPA+ACI, 10 mM of AMPKI or 10 mM of AICAR, followed by culture for 1, 2, and 4 days. To analyze cell cycle, the cells were washed with buffer twice and then the cells were centrifuged using 0.25% trypsin, followed by fixation in cold 70% ethanol. Analysis was performed by flow cytometry (Becton Dickinson FACSorter) using 50 mg/ml of PI containing RNase.

7. Statistical Analysis

Statistical analysis was performed using Graph-Pad Prism (GraphPad, San Diego, Calif.). T-test was performed for verification for the comparison between the LPA treated group and the LPA untreated group (1 day/2 day/4 day). At this time, significance level was set 0.001. Thus, when p<0.001, it was judged as statistically significant.

<Results>

1. LPA and ACI increase senescent cell proliferation and promote S phase entry.

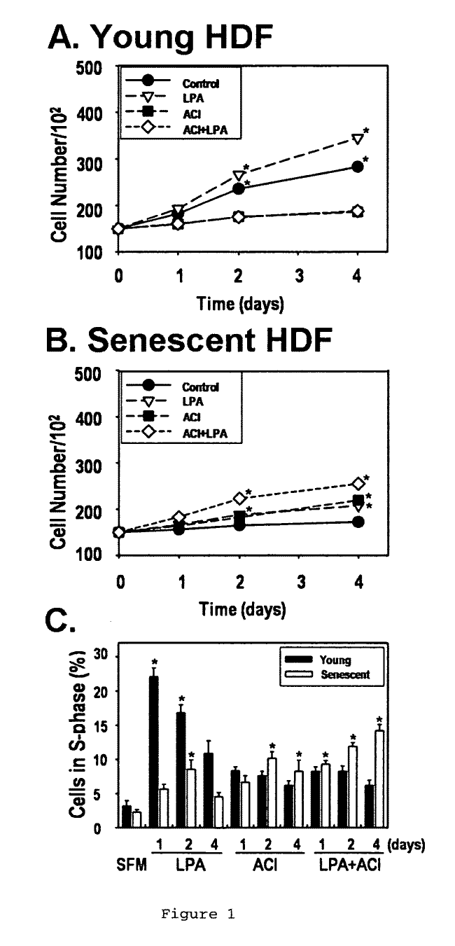

Cell proliferation and cAMP level were increased by LPA in senescent cells (Yeo et al., 2002). The AC inhibitor (ACI) was added to reduce cAMP which had been increased by LPA. And then, cell proliferation was investigated. Cells were cultured in serum-free medium for 2 days to arrest them in G0/G1 phase. Then, the cells were treated with LPA, ACI or LPA+ACI. Total cell number was measured on day 1, day 2 and day 4, or the cells entered into S phase were counted to evaluate cell proliferation. When young cells were treated with 300 μM of ACI, cell proliferation was reduced, compared with control (FIG. 1A). In the meantime, when senescent cells were treated with 300 μM of ACI, cell proliferation was increased, compared with control (FIG. 1B). When LPA was treated to young cells, cell proliferation was increased, whereas when LPA and ACI were treated simultaneously to young cells, cell proliferation was completely inhibited. However, when LPA and ACI were treated simultaneously to senescent cells, cell proliferation was significantly increased compared with when LPA or ACI was treated alone (FIG. 1B, ACI+LPA).

The number of cells entered into S phase was measured and the result shows a similar response to LPA and ACI. In senescent cells, not only co-treatment of LPA and ACI but also ACI single treatment increased the cell number entered into S phase (FIG. 1C). The above results indicate that the effect of ACI in young cells was different from that in senescent cells. That is, only LPA increases cell proliferation in young cells, but both LPA and ACI can increase cell proliferation in senescent cells.

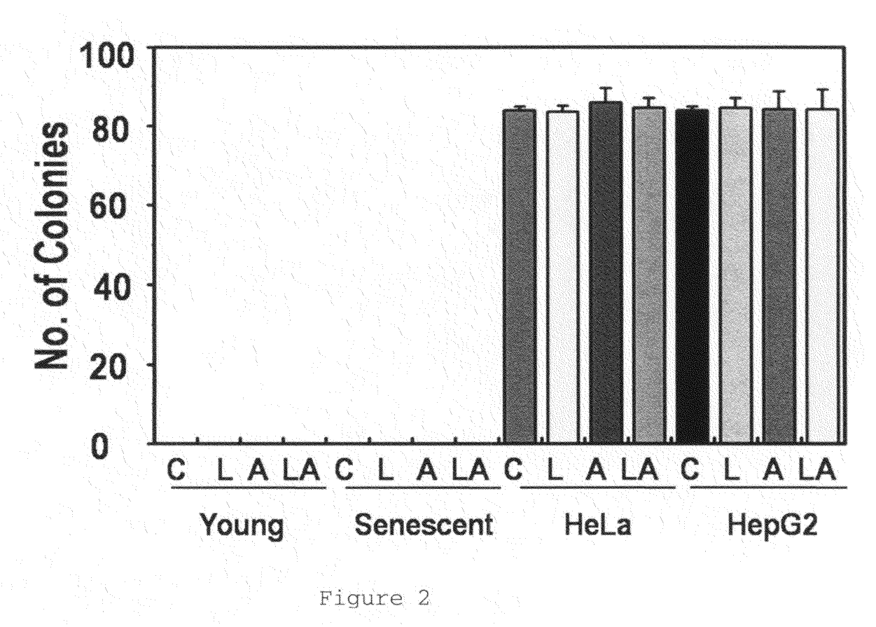

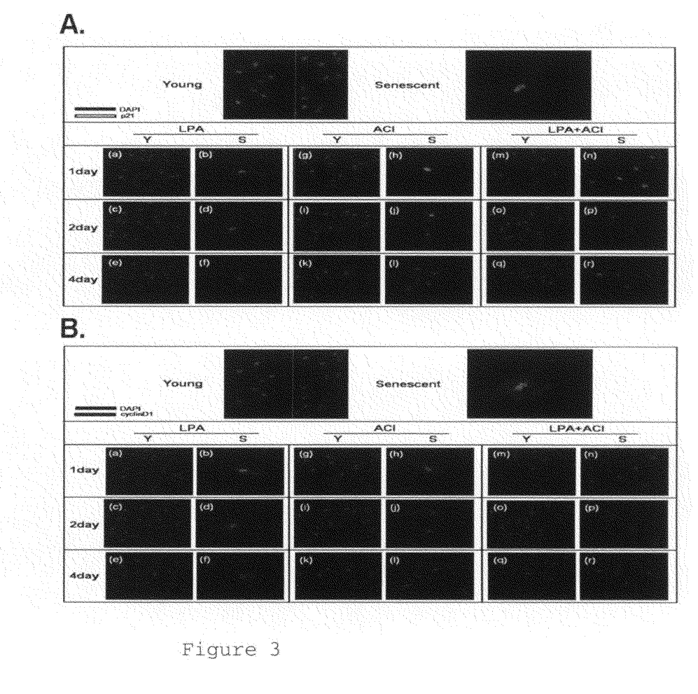

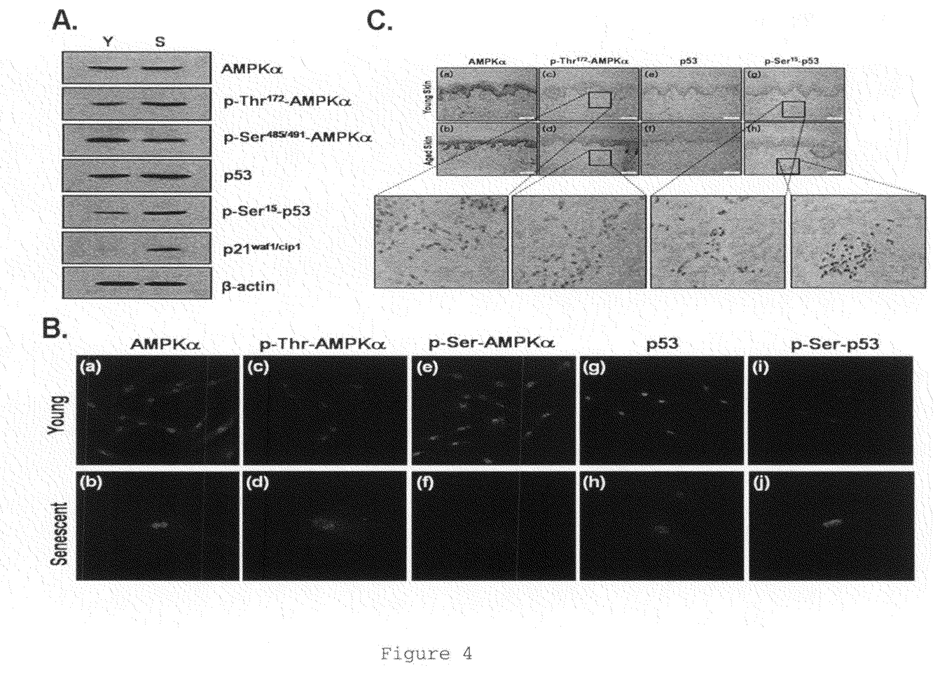

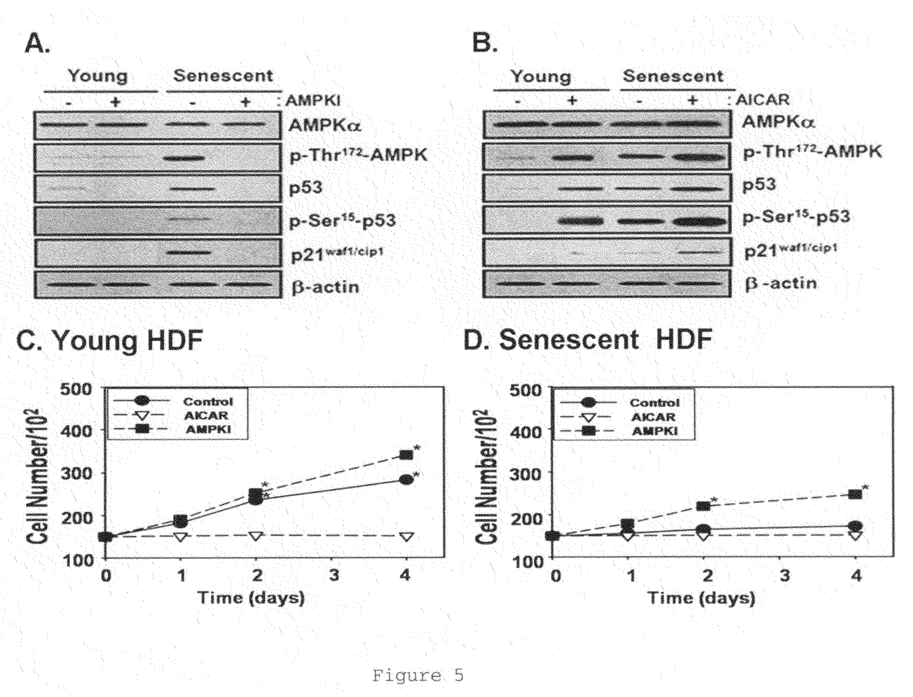

LPA and ACI were treated to fibroblast and cancer cell groups, followed by soft agar assay. As a result, unlike in cancer cell lines, LPA or ACI treatment did not form any colony in fibroblasts (FIG. 2). From the above results, it was confirmed that LPA and ACI induce normal cell proliferation but not cause any transformation of cells to turn them into tumor.

2. Down-Regulation of p21 and Cyclin D1 by LPA and ACI in Senescent Cells

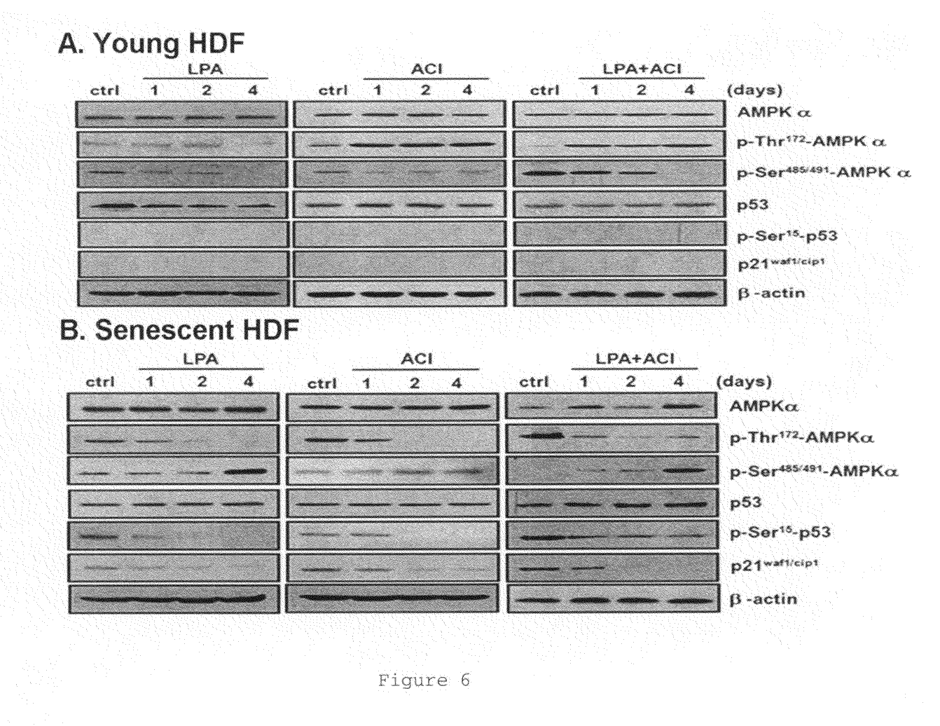

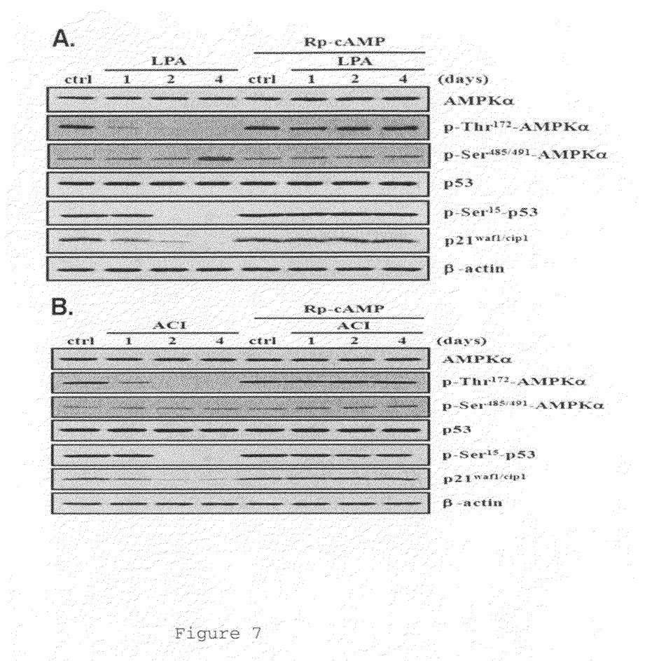

P21 and cyclin D1 are important proteins for maintaining pRb in the hypophosphorylated forms (Noda et al., 1994), which have been known to suppress cell proliferation and prohibit cells from advancing to S phase (Atadja et al., 1995; Stein et al., 1999) and are significantly upregulated in senescent cells. Cells were treated with 30 μM of LPA, 300 μM of ACI or LPA+ACI for 4 days, followed by immunofluorescence to investigate p21 and cyclin D1 expressions (FIG. 3A). As a result, when young cells were treated with ACI alone or LPA+ACI, p21 expression was increased on day 2 and day 4 (FIG. 3A).

In the meantime, when senescent cells were treated with ACI, p21 expression was reduced on day 2 and day 4. When senescent cells were treated with ACI, most cells were changed into young cell like cells on day 4. Microscope observation also confirmed that young cell like cells were increased compared with the control (untreated senescent cells are bigger in size, so that less cells can be observed under microscope, compared with treated senescent cells). When senescent cells were treated with LPA and ACI simultaneously, p21 expression was significantly reduced, compared with when they were treated with ACI alone. So was cyclin D1 expression (FIG. 3B). These results indicate that the elevation of p21 and cyclin D1 correlate with entering the S phase. Thus, when senescent cells were treated with ACI, p21 and cyclin D1 expressions were reduced and thereby DNA synthesis in senescent cell increased to induce cell proliferation.

3. AMPK Activity in Senescent Cells and Back Skin Cells of Aged Man

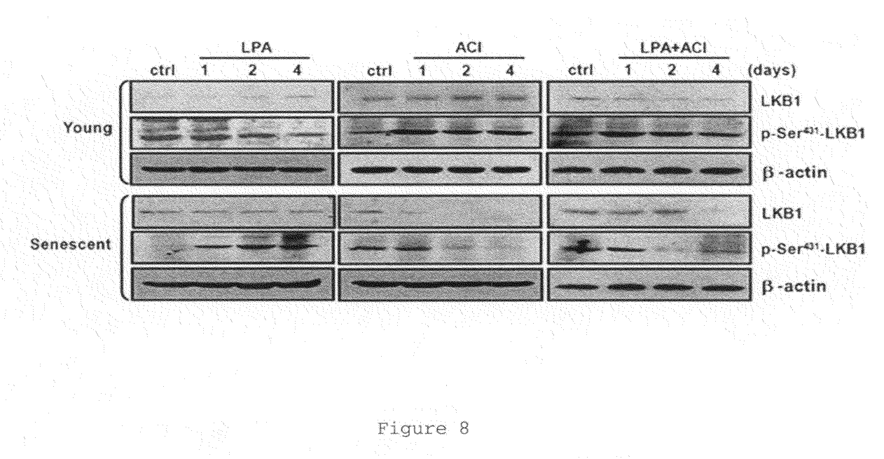

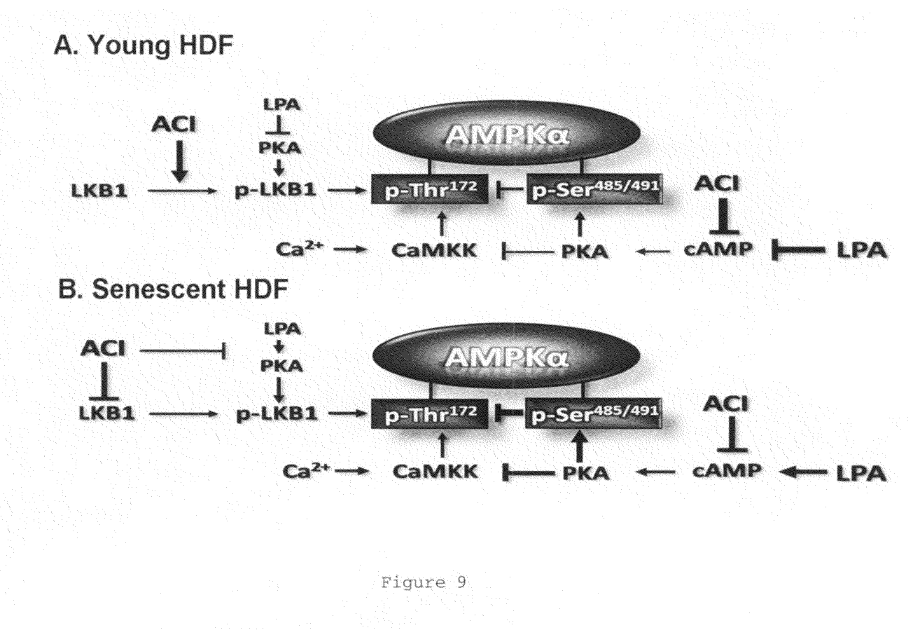

In cellular senescence, it is well known that the increase of the ratio of AMP:ATP induces AMPK activation (Wang et al., 2003). P53 is the activated AMPK substrate. AMPK induces Ser15 phosphorylation, which is essential for p21 expression (Jones et al., 2005). In this example, phosphorylation of Thr172-AMPKα exhibiting AMPKα activity was confirmed by immuno-blotting in order to compare AMPK activity between young cells and senescent cells (FIG. 4A). Phosphorylation of Ser485/491-AMPK that reduces AMPK activity, and p53, p-Ser15-p53, p21 and β-actin were also measured by immuno-blotting. As a result, expressions of p-Thr172-AMPKα, p53, p-Ser15-p53 and p21 were low in young cells. But, in senescent cells, phosphorylation of Thr172-AMPKα, the activated form of AMPK, was increased, while phosphorylation of Ser485/491-AMPKα, the inactivated form of AMPK, was reduced. However, the total amount of AMPK was not changed as being aged. Phosphorylation of p53 on Ser15 and the expression of p21 was increased in senescent cells, suggesting that AMPK was activated therein.

The expressions of p-Thr172-AMPKα, p-Ser485/491-AMPKα, and p-Ser15-p53 in both young and senescent cells were investigated by confocal microscope (FIG. 4B). AMPK was mostly found in cytoplasm regardless of phosphorylation, but sometimes found in nucleus. Phosphorylation of Thr172-AMPKα in senescent cells was increased, compared with that in young cells. But, phosphorylation of Ser485/491-AMPKα was reduced in senescent cells, compared with in young cells. P53 was mostly found in cytoplasm but phosphorylation of Ser15-p53 was detected in nucleus of senescent cell.

It was confirmed by immuno-staining of back skin tissues of both young and aged people that AMPK phosphorylation and activation were increased not only in young cells but also in senescent cells (FIG. 4C). There was no difference in expressions of AMPKα and p53 between young and aged back skin tissues. However, p-Thr172-AMPKα was increased in aged back skin tissues, while p-Ser15-p53 was increased in young back skin tissues. The above results indicate that activated AMPK and p53 expression were increased in aged subjects and mostly found in nucleus.

4. Senescent Cell Proliferation is Regulated by AMPK Activation

To investigate whether AMPK activation could inhibit senescent cell proliferation, AMPK activation inhibitor AMPKI and AMPK activation promoter AICAR were treated to cells (FIG. 5). Then, p-Thr172-AMPKα, p-Ser15-p53 and p21 expressions therein were measured. AMPKα, p53 and β-actin were used as controls. AMPKI did not affect expressions of p-Thr172-AMPKα, p-Ser15-p53 and p21 in young cells (FIG. 5A). But, expression levels of p-Ser15-p53 and p21 were low in young cells. AMPKI completely abrogated the elevation of those proteins in senescent cells. Unlike AMPKI, AICAR rather increased those proteins in senescent cells (FIG. 5B). This suggests that AMPK increases p21 activity in senescent cells, so that cell proliferation is reduced thereby and AICAR increases AMPK in young cells and reduces cell proliferation and AMPKI inhibits AMPK in senescent cells and reduces p21 expression, so that cell proliferation is increased.

As shown in FIG. 5C and FIG. 5D, AMPKI increased cell proliferation in both young and senescent cells. In the meantime, AICAR suppressed cell proliferation in both cells. Therefore, when AMPK is activated, cell proliferation is inhibited in both young and senescent cells. So, it became clear that AMPKI promoted senescent cell proliferation (FIG. 5D) by the decrease of Ser15-p53 phosphorylation and p21 expression mediated by AMPK inactivation. On the other hand, AICAR inhibited cell proliferation of both young and senescent cells by the increase of p53 phosphorylation and p21 expression mediated by AMPK activation.

5. Different AMPK Phosphorylation Patterns by LPA and ACI in Young and Senescent Cells

LPA and ACI increased senescent cell proliferation. And also, these substances were confirmed in this example to have an effect on AMPK phosphorylation to control its activity. When LPA was treated to young cells, phosphorylation of Thr172-AMPKα and Ser485/491-AMPKα was all reduced on day 4 (FIG. 6A). LPA treatment did not change β-actin (control) and AMPK levels. Levels of p-Ser15-p53 and p21 could not be detected (basically expressions of these proteins are very low in young cells). When LPA was treated to senescent cells, phosphorylation of Thr172-AMPKα was reduced on day 4, but phosphorylation of Ser485/491-AMPKα was gradually increased (FIG. 6B). It was also confirmed that when LPA was treated to senescent cells, expressions of p-Ser15-p53 and p21 were reduced.

As shown in the above, when LPA was treated to young cells, AMPK activity was decreased but can still be detected until day 4. But, when LPA was treated to senescent cells, AMPK activity was gradually reduced and almost inhibited until day 4. LPA was also confirmed to increase cell proliferation in both young and senescent cells. In the meantime, when ACI was treated to young cells, Thr172-AMPKα phosphorylation began to increase as a day passed, but Ser485/491-AMPKα phosphorylation was reduced.

The expressions of Ser15-p53 and p21 could not be confirmed in young cells (their expressions are basically very low in young cells). In senescent cells, ACI did not affect Ser485/491-AMPKα phosphorylation, but reduced Thr172-AMPKα phosphorylation. In addition, when ACI was treated to senescent cells, Ser15-p53 phosphorylation and p21 expression were reduced.

As described hereinbefore, in young cells, ACI increases AMPK activity and thus inhibits cell proliferation. But, in senescent cells, ACI reduces AMPK activity and thus increases cell proliferation.

When LPA and ACI were co-treated to young cells, same protein expression patterns were observed as those under ACI single treatment. Precisely, when LPA and ACI were co-treated to young cells, Thr172-AMPKα phosphorylation was increased, but when they were co-treated to senescent cells, the phosphorylation was reduced. Therefore, it was confirmed that the increase of senescent cell proliferation was caused by the decrease of AMPK activity, resulting in the decrease of p53 phosphorylation and p21 expression.

As described hereinbefore, inhibition of AMPK activity is important to increase senescent cell proliferation. And, this can be achieved by regulating phosphorylation of various regions of AMPK.

6. PKA Involved in AMPK Inhibition by LPA in Senescent Cells

In previous study, it was confirmed that PKC dependent AC isotype (AC2/4/6) expression was increased in senescent cells so as to increase its activity and as a result cAMP was up-regulated to increase cAMP dependent kinase PKA activity (Jang et al., 2006b; Rhim et al., 2006). Besides, Ser485/591 phosphorylation playing a certain role in inhibiting AMPK activity was regulated by PKA activity (Hurley et al., 2006), and PKA mediated Ser485/591 phosphorylation inhibited Thr172-AMPKα phosphorylation in the end. Based on that, it was further investigated whether PKA signal transduction played an important role in senescent cells as well. To do so, PKA inhibitor Rp-cAMP was pre-treated to senescent cells for one hour before the experiment (FIG. 7).

As shown in FIG. 7A, when senescent cells were treated with LPA after suppressing PKA, the expressions of p-Thr172-AMPKα, p-Ser485/491-AMPKα, p-Ser15-p53 and p21 were not changed at all. This result suggests that PKA plays an important role in upstream signal transduction mediated by an increase of ser485/491 phosphorylation in relation to regulation of LPA mediated AMPK activity.

As shown in FIG. 7B, when senescent cells were treated with ACI after suppressing PKA, the expression of p-Ser485/491 was not changed and the expressions of p-Thr172-AMPKα, p-Thr172-AMPKα, p-Ser15-p53 and p21 were not changed, either.

The above results indicate that ACI plays a certain role in blocking downstream signal transduction by PKA. That is, PKA also plays an important role in regulation of ACI mediated AMPK activity in senescent cells.

7. Phosphorylation Status on Ser431 of the Tumor Suppressor, Serine/Threonine Protein Kinases LKB1, is Regulated by LPA and LKB1 Protein Expression is Reduced by ACI in Senescent Cells.

It has been recently discovered that the tumor suppressor gene LKB1 is a member of AMPKK family (Hawley et al., 2003; Shaw et al., 2004; Woods et al., 2003a). Ser431-LKB1 phosphorylation promotes cell growth in the activated form of LKB1 (Sapkota et al., 2001). So, in this experiment, it was examined what effect the single treatment or co-treatment of LPA and ACI has on the expressions of p-Ser431-LKB1, LKB1 and β-actin (FIG. 8). When LPA was treated to young cells, Ser431-LKB1 phosphorylation was gradually reduced. Meanwhile, LKB1 expression therein was not changed. On the other hand, when LPA was treated to senescent cells, Ser431-LKB1 phosphorylation was gradually increased.

ACI increased Ser431-LKB1 phosphorylation in young cells, while it reduced LKB1 and Ser431-LKB1 phosphorylation gradually in senescent cells. Co-treatment of LPA and ACI produced the same result as obtained from the ACI single treatment.

<Discussion>

When senescent cells were treated with LPA, cAMP was up-regulated. Thus, the effect of cAMP on cell proliferation in senescent cells was investigated by down-regulating cAMP with the AC inhibitor (SQ22536). Interestingly, ACI completely inhibited PKA activity in both young and senescent cells. And, while ACI reduced the number of young cells, it increased the number of senescent cells. ACI reduced the expressions of p21 and cyclin D1, two cell cycle inhibitors, in senescent cells, by which it was presumed that the number of cells entering S phase was increased (Atadja et al., 1995; Stein et al., 1999). It was also confirmed that ACI changed numbers of senescent cells into young cell like cells. When LPA and ACI were co-treated to young cells, we could not observe such phenomena as observed when LPA was treated alone, for example; increase of cell proliferation, promotion of S phase entry, and decrease of p21 and cyclin D1 expressions. In the meantime, when LPA and ACI were co-treated to senescent cells, cell proliferation was more effectively induced, compared with LPA or ACI was treated alone. It was presumed that the increase of DNA synthesis and cell proliferation in senescent cells were resulted from ACI mediated reduction of p21 and cyclin D1 expressions. In this experiment, it was confirmed that LPA induced cell proliferation in both young and senescent cells, while ACI inhibited cell proliferation in young cells but increased cell proliferation in senescent cells.

AMPK inhibits cell proliferation by regulating various cellular events in both normal and tumor cells (Motoshima et al., 2006). And, AMPK is activated when cells are aged (Wang et al., 2003). It was proposed that AMPK activity might inhibit cell cycle by controlling p21 expression and Ser15 phosphorylation of p53 in senescent cells (Jones et al., 2005). Thus, continuous inducement of AMPK activation accelerates p53 dependent cellular senescence. This experiment was performed based on the hypothesis that when LPA or ACI is treated to fibroblasts, it regulates AMPK activity to control cell proliferation. And as a result, it was confirmed that AMPK activation, evaluated by Thr172-AMPKα phosphorylation, Ser15 phosphorylation of p53, and p21 expression were all increased in senescent cells and in back skin tissues of aged people.

LPA was confirmed to reduce AMPK activation in both young and senescent cells. Such decrease of AMPK activation might play a certain role in the increase of LPA dependent cell proliferation in young and senescent cells. In senescent cells, LPA reduced the expressions of p-Ser15-p53 and p21 so as to release cell cycle arrested in G0/G1 phase. When ACI was treated alone or together with LPA to young cells, AMPK activation therein was increased. On the contrary, such treatment reduced AMPK activation in senescent cells, suggesting that ACI reduced young cell proliferation but increased senescent cell proliferation. This experiment confirmed that LPA and ACI regulated AMPK activation differently in young and senescent cells, so that they affected cell proliferation differently.

AMPK activity can be regulated by multisite phosphorylation by several AMPKK (Hurley et al., 2006). To confirm the hypothesis that LPA and ACI regulate multisite phosphorylation of AMPK differently, the activated AMPK form, phosphorylated Thr172-AMPKα and the inactivated AMPK form, phosphorylated Ser485/491-AMPKα levels were measured. LPA reduced Thr172-AMPKα phosphorylation that activated AMPK in young and senescent cells, while ACI increased Thr172-AMPKα phosphorylation to activate AMPK in young cells but reduced the phosphorylation to inactivate AMPK in senescent cells. Thus, it was confirmed that LPA and ACI regulated AMPK activity in different way, so that their effects on cell proliferation in young and senescent cells were also different. Ser485/491 phosphorylation inhibits Thr172 phosphorylation. Therefore, it was suggested that when Ser485/491 phosphorylation was increased by LPA, AMPK activity in senescent cells was reduced.

When ACI was treated singly or together with LPA to young cells, Ser485/491-AMPKα phosphorylation was reduced, resulting in the increase of AMPK activity, suggesting that cell proliferation was inhibited in young cells. When ACI was treated to senescent cells, Ser485/491-AMPKα phosphorylation was not changed, but ACI itself inhibited Thr172 phosphorylation so that AMPK activity was reduced in the end. Such results indicate that ACI has different mechanism of inhibiting AMPK activity in senescent cells.

Unlike LPA, it is believed that ACI regulates AMPKK to control AMPK activity and cell proliferation thereby. Under severe energy deficiency or other tough conditions, ACI activates LKB1 to induce Thr172-AMPK phosphorylation (Hawley et al., 2003; Shaw et al., 2004; Woods et al., 2003a). Thr172-AMPK phosphorylation can also be increased by calcium/calmodulin enzyme when intracellular calcium level is increased, resulting in AMPK activation as well (Hawley et al., 2005; Hong et al., 2005; Hurley et al., 2005; Woods et al., 2005). The auto-phosphorylation site of AMPK, Ser485/491, also plays a certain role in inhibiting AMPK activation by foreign stimuli or intracellular energy deficiency (Hurley et al., 2006). Ser485/591 site is also phosphorylated by Akt/PKB activated by insulin stimulus (Beauloye et al., 2001 Gamble and Lopaschuk, 1997; Kovacic et al., 2003; Witters and Kemp, 1992) and also by PKA activated by those drugs that increase cAMP (Hurley et al., 2006). This experiment was focused on two protein phosphorylation kinases, PKA and LKB1, among many upstream signals.

LKB1 forms a complex with co-proteins such as STRAD (STE20-related adaptor) α/β and MO25 (mouse protein 25) α/β, and this complex increases LKB1 activity. The LKB1/STRAD/Mo25 complex is known as a kinase existing in upstream of AMPK/TSC2/mTOR pathway (Hawley et al., 2003; Milburn et al., 2004). LKB1 activity is regulated by Ser431 phosphorylation, as well-known already, and phosphorylations of four other different regions (Ser³¹, Ser³²⁵, Thr³³⁶ and Thr³⁶⁶) (Sapkota et al., 2002; Sapkota et al., 2001). Basically in young cells, Ser431-LKB1 phosphorylation is increased, compared with in senescent cells, resulting in LKB1 activation. The activated LKB1 can be a reason for the block of cell proliferation in young cells that are arrested in resting phase by the activation of AMPK and p53 and the increased expression of p21 thereby. When LPA was treated to young cells, the level of LKB1 itself was not affected, but the level of phosphorylated Ser431-LKB1 was gradually reduced. The decrease of LKB1 activity resulted in the decrease of Thr172-AMPKα phosphorylation in young cells, leading to the decrease of AMPK activity. In the case of ACI, it increased Ser431-LKB1 phosphorylation and thereby increases the level of phosphorylated Thr172-AMPKα in young cells. ACI inactivated PKA in young cells and thereby reduced PKA dependent Ser485/491α phosphorylation. Interestingly, unlike in young cells, ACI reduced both the levels of total LKB1 and phosphorylated LKB1 in senescent cells. The inhibition of LKB1 phosphorylation might result in the suppression of p-Thr172-AMPKα, p-Ser15-p53 and p21 expressions.

ACI inactivated PKA in senescent cells. And as a result, PKA dependent Ser485/491-AMPK phosphorylation and Ser431-LKB1 phosphorylation were also reduced. When LPA and ACI were co-treated to cells, LKB1 and LKB1 phosphorylation patterns were similar to those under ACI single treatment, but the effect was smaller than when ACI was treated alone.

PKA is an upstream kinase that directly induces phosphorylation of Ser485/491-AMPK (Hurley et al., 2006) or indirectly induces Thr172-AMPK phosphorylation via LKB1 phosphorylation (Collins et al., 2000; Sapkota et al., 2001). Thus, AMPK activation by LKB1 phosphorylation can be regulated by the control of PKA activation by LPA and ACI. When LPA was treated to young cells, cAMP was down-regulated, and thereby PKA activity was reduced (Jang et al., 2006b). That is, LPA reduced Ser485/491-AMPKα phosphorylation in young cells. However, LPA activated PKA in senescent cells (Jang et al., 2006a). So, PKA dependent Ser485/491-AMPKα phosphorylation was also increased and thereby Thr172-AMPKα phosphorylation was reduced. When PKA inhibitor was treated to senescent cells, the change in the expressions of p-Thr172-AMPKα, p-Ser485/491-AMPKα, p-Ser15-p53 and p21 induced by LPA were all blocked completely. This suggests that PKA could be a major upstream protein that inactivates AMPK via an increase of Ser485/491-AMPKα phosphorylation. In conclusion, LPA reduces Ser431-LKB1 phosphorylation in young cells but increases that in senescent cells. So, LKB1 dependent Thr172-AMPKα phosphorylation is reduced by LPA in young cells but it is increased in senescent cells. When PKA inhibitor was treated to senescent cells, PKA was inactivated and thus a reduction of expressions of p-Thr172-AMPKα, p-Ser15-p53 and p21 induced by ACI were blocked, indicating that PKA is one of important upstream proteins involved in ACI dependent AMPK inactivation. PKA phosphorylates another upstream kinase CaMKKs, which results in the inhibition of AMPKK activity, suggesting that it indirectly regulates Thr172-AMPKα phosphorylation. Therefore, AMPK activity can be regulated by the changes of PKA, LKB1 and CaMKKs activities as a whole. PKA (Cohen and Hardie, 1991) and AMPK (Kahn et al., 2005; Long and Zierath, 2006) can be activated not only by hormonal stimulation via β-adrenergic receptors but also by physiological stimuli such as exercise and fasting.

AMPK signal transduction system includes many tumor suppressor genes such as LKB1, p53, TSC1 or TSC2, which are acting as metabolic regulation switches to inhibit signal transduction of growth factors caused by various stimuli. Previous studies point out that AMPK activation can be a target of treating aging-related disease rooted in cellular senescence and proliferation such as arteriosclerosis, insulin tolerance and cancer (Igata et al., 2005; Luo et al., 2005; Motoshima et al., 2006; Shaw et al., 2004). AICAR mediated AMPK activation induces cell cycle arrest in normal cells such as human vascular smooth muscle cells or cancer cells. In vascular smooth muscle cells, AICAR increases p53 protein level and Ser15-p53 phosphorylation and thereby the cells are arrested in Go/G1 phase, suggesting that the number of cells entering S or G2/M phase is reduced (Igata et al., 2005). In cancer cell, AICAR arrests cells in S phase, so that along with the increased expressions of p21, p27 and p53, AICAR inhibits tumor cell proliferation (Rattan et al., 2005). This experiment confirmed that AICAR inhibited cell proliferation in both young and senescent cells by activating AMPK. AICAR also increased expressions of p-Thr172-AMPKα, p53, p-Ser15-p53 and p21 in young and senescent cells, resulting in the inhibition of cell proliferation. In the meantime, AMPKI increased cell proliferation in young and senescent cells. When AMPK activation was suppressed by the treatment of AMPKI in senescent cells, the expressions of p-Thr172-AMPKα, p53, p-Ser15-p53 and p21 were reduced, so that not only cell proliferation but also morphological change into young cell like cells were observed. Therefore, it was confirmed that inhibition of AMPK activation was essential to prevent cellular senescence by LPA and ACI.

In conclusion, from this experiment, it was provided a model illustrating that LPA and ACI regulates AMPK activity differently in senescent cells (FIG. 9). The active a subunit of AMPK contains two major phosphorylation sites, which are α-Thr172 and α-Ser485/491. When Thr172-AMPKα is phosphorylated, AMPK activity is increased, while when Ser485/491-AMPKα is phosphorylated, Thr172-AMPKα phosphorylation is reduced and thus AMPK activity is suppressed. When LPA is treated to young cells, intracellular cAMP is down-regulated and PKA is inhibited, resulting in the decrease of Ser485/491-AMPKα phosphorylation (FIG. 9A). However, in young cells, LPA reduces PKA dependent LKB1 phosphorylation and thus reduces Thr172-AMPKα phosphorylation. As a result, AMPK is inactivated and cell proliferation is increased. ACI suppresses cAMP/PKA signal transduction system and thereby reduces Ser485/491-AMPKα phosphorylation, resulting in AMPK activation. Also, ACI increases LKB1 activity slightly, and thus Thr172-AMPKα phosphorylation is induced to activate AMPK. When LPA is treated to senescent cells, intracellular cAMP is up-regulated and PKA is activated and thereby Ser485/491-AMPKα phosphorylation is increased but Thr172-AMPKα phosphorylation is reduced, resulting in AMPK inactivation and the increase of cell proliferation (FIG. 9B). On the contrary, ACI dose not change Ser485/491-AMPKα phosphorylation, but mediates the decrease of Thr172-AMPKα phosphorylation via LKB1 expression decrease, so that it inactivates AMPK in the end and thus induces cell proliferation. This invention confirms that not only young cells but also senescent cells have cell proliferation capacity and LPA and ACI regulates phosphorylation of various sites of AMPK differently to inhibit AMPK activity, which can induce senescent cell proliferation.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

### REFERENCE

1. An S, Bleu T, Hallmark O G, Goetzl E J (1998) Characterization of a novel subtype of human G protein-coupled receptor for lysophosphatidic acid. J Biol Chem 273, 7906-7910.

2. Atadja P, Wong H, Yeillete C, Riabowol K (1995) Overexpression of Cyclin D1 Blocks Proliferation of Normal Diploid Fibroblasts. Experimental Cell Research 217, 205.

3. Beauloye C, Marsin A S, Bertrand L, Krause U, Hardie D G., Vanoverschelde J L, Hue L (2001) Insulin antagonizes AMP-activated protein kinase activation by ischemia or anoxia in rat hearts, without affecting total adenine nucleotides. FEBS Letters 505, 348.

4. Boyce S T, Ham R G (1983) Calcium-regulated differentiation of normal human epidermal keratinocytes in chemically defined clonal culture and serum-free serial culture. J Invest Dermatol 81, 33s-40s.

5. Cohen P, Hardie D G (1991) The actions of cyclic AMP on biosynthetic processes are mediated indirectly by cyclic AMP-dependent protein kinase. Biochim Biophys Acta 1094, 292-299.

6. Collins S P, Reoma J L, Gamm D M, Uhler M D (2000) LKB1, a novel serine/threonine protein kinase and potential tumour suppressor, is phosphorylated by cAMP-dependent protein kinase (PKA) and prenylated in vivo. Biochem J 345 Pt3, 673-680.

7. Gamble J, Lopaschuk G D (1997) Insulin inhibition of 5′ adenosine monophosphate—activated protein kinase in the heart results in activation of acetyl coenzyme A carboxylase and inhibition of fatty acid oxidation. Metabolism 46, 1270.

8. Hahn-Windgassen A, Nogueira V, Chen C C, Skeen J E, Sonenberg N, Hay N (2005) Akt activates the mammalian target of rapamycin by regulating cellular ATP level and AMPK activity. J Biol Chem 280, 32081-32089.

9. Hawley S A, Boudeau J, Reid J L, Mustard K J, Udd L, Makela T P, Alessi D R, Hardie D G (2003) Complexes between the LKB1 tumor suppressor, STRAD alpha/beta and MO25 alpha/beta are upstream kinases in the AMP-activated protein kinase cascade. J Biol 2, 28.

10. Hawley S A, Pan D A, Mustard K J, Ross L, Bain J, Edelman A M, Frenguelli B G, Hardie D G (2005) Calmodulin-dependent protein kinase kinase-[beta] is an alternative upstream kinase for AMP-activated protein kinase. Cell Metabolism 2, 9.

11. Hong, S P, Momcilovic M, Carlson M (2005) Function of Mammalian LKB1 and Ca2+/Calmodulin-dependent Protein Kinase Kinase α as Snf1-activating Kinases in Yeast. J Bio Chem 280, 21804-21809.

12. Horman S, Vertommen D, Heath R, Neumann D, Mouton V, Woods A, Schlattner, U, Wallimann T, Carling D, Hue L, Rider M H (2006) Insulin Antagonizes Ischemia-induced Thr172 Phosphorylation of AMP-activated Protein Kinase α-Subunits in Heart via Hierarchical Phosphorylation of Ser485/491. J Biol Chem 281, 5335-5340.

13. Hurley R L, Anderson K A, Franzone J M, Kemp B E, Means A R, Witters L A (2005) The Ca2+/Calmodulin-dependent Protein Kinase Kinases Are AMP-activated Protein Kinase Kinases. J Biol Chem 280, 29060-29066.

14. Hurley R L, Barre L K, Wood S D, Anderson K A, Kemp B E, Means A R, Witters L A (2006) Regulation of AMP-activated Protein Kinase by Multisite Phosphorylation in Response to Agents That Elevate Cellular cAMP. J Biol Chem 281, 36662-36672.

15. Igata M, Motoshima H, Tsuruzoe K, Kojima K, Matsumura T, Kondo T, Taguchi T, Nakamaru K, Yano M, Kukidome D (2005) Adenosine monophosphate-activated protein kinase suppresses vascular smooth muscle cell proliferation through the inhibition of cell cycle progression. Circ Res 97, 837-844.

16. Jang I S, Rhim J H, Kim K T, Cho K A, Yeo E J, Park S C (2006a) Lysophosphatidic acid-induced changes in cAMP profiles in young and senescent human fibroblasts as a clue to the ageing process. Mech Ageing Dev 127, 481-489.

17. Jang I S, Rhim J H, Park S C, Yeo E J (2006b) Downstream molecular events in the altered profiles of lysophosphatidic acid-induced cAMP in senescent human diploid fibroblasts. Exp Mol Med 38, 134-143.

18. Jang I S, Yeo E J, Park J A, Ahn J S, Park J S, Cho K A, Juhnn Y S, Park S C (2003) Altered cAMP signaling induced by lysophosphatidic acid in senescent human diploid fibroblasts. Biochem Biophys Res Commun 302, 778-784.

19. Jones R G, Plas D R, Kubek S, Buzzai M, Mu J, Xu Y, Birnbaum M J, Thompson C B (2005) AMP-activated protein kinase induces a p53-dependent metabolic checkpoint. Mol Cell 18, 283-293.

20. Kahn B B, Alquier T, Carling D, Hardie D G. (2005) AMP-activated protein kinase: Ancient energy gauge provides clues to modern understanding of metabolism. Cell Metabolism 1, 15.

21. Kovacic S, Soltys C L M, Barr A J, Shiojima I, Walsh K, Dyck J R B (2003) Akt Activity Negatively Regulates Phosphorylation of AMP-activated Protein Kinase in the Heart. J Biol Chem 278, 39422-39427.

22. Long Y C, Zierath J R (2006) AMP-activated protein kinase signaling in metabolic regulation. J Clin Invest 116, 1776-1783.

21. Luo Z, Saha A K, Xiang X, Ruderman N B (2005) AMPK, the metabolic syndrome and cancer. Trends Pharmacol Sci 26, 69-76.

22. Milburn C C, Boudeau J, Deak M, Alessi D R, Aalten D M (2004) Crystal structure of MO25 alpha in complex with the C terminus of the pseudo kinase STE20-related adaptor. Nat Struct Mol Bio 111, 193-200.

23. Mitchelhill K I, Michell B J, House C M, Stapleton D, Dyck J, Gamble J, Ullrich C, Witters L A, Kemp B E (1997) Posttranslational Modifications of the 5′-AMP-activated Protein Kinase beta 1 Subunit. J Biol Chem 272, 24475-24479.

24. Moolenaar W H (2000) Development of our current understanding of bioactive lysophospholipids. Ann NY Acad Sci 905, 1-10.

25. Moolenaar W H, Kranenburg O, Postma F R, Zondag G C (1997) Lysophosphatidic acid: G-protein signalling and cellular responses. Curr Opin Cell Biol 9, 168-173.

26. Motoshima H, Goldstein B J, Igata M, Araki E (2006) AMPK and cell proliferation-AMPK as a therapeutic target for atherosclerosis and cancer. J Fhysiol 574, 63-71.

27. Noda A, Ning Y, Venable S F, Pereira-Smith O M, Smith J R (1994) Cloning of Senescent Cell-Derived Inhibitors of DNA Synthesis Using an Expression Screen. Experimental Cell Research 211, 90.

28. Peacocke M, Campisi J. (1991) Cellular senescence: a reflection of normal growth control, differentiation, or aging? J Cell Biochem. 45, 147-55.

29. Rattan R, Giri S, Singh A K, Singh I (2005) 5-Aminoimidazole-4-carboxamide-1-beta-D-ribofuranoside inhibits cancer cell proliferation in vitro and in vivo via AMP-activated protein kinase. J Biol Chem 280, 39582-39593.

30. Rhim J H, Jang I S, Yeo E J, Song K Y, Park S C (2006) Role of protein kinase C-dependent A-kinase anchoring proteins in lysophosphatidic acid-induced cAMP signaling in human diploid fibroblasts. Aging Cel 15, 451-461.

31. Sapkota G P, Boudeau J, Deak M, Kieloch A, Morrice N, Alessi D R (2002) Identification and characterization of four novel phosphorylation sites (Ser31, Ser325, Thr336 and Thr366) on LKB1/STK11, the protein kinase mutated in Peutz-Jeghers cancer syndrome. Biochem J 362, 481-490.

32. Sapkota G P, Kieloch A, Lizcano J M, Lain S, Arthur J S C, Williams M R, Morrice N, Deak M, Alessi D R (2001) Phosphorylation of the Protein Kinase Mutated in Peutz-Jeghers Cancer Syndrome, LKB1/STK11, at Ser431 by p90RSK and cAMP-dependent Protein Kinase, but Not Its Farnesylation at Cys433, Is Essential for LKB1 to Suppress Cell Growth. J Biol Chem 276, 19469-19482.

33. Shaw R J, Kosmatka M, Bardeesy N, Hurley R L, Witters L A, DePinho R A, Cantley L C (2004) The tumor suppressor LKB1 kinase directly activates AMP-activated kinase and regulates apoptosis in response to energy stress. Proc Natl Acad Sci 101, 3329-3335.

34. Smith J R, Pereira-Smith O M. (1996) Replicative senescence: implications for in vivo aging and tumor suppression. Science 273, 63-67.

35. Soltys C L M, Kovacic S, Dyck J R B (2006) Activation of cardiac AMP-activated protein kinase by LKB1 expression or chemical hypoxia is blunted by increased Akt activity. Am. J. Physiol. Heart Circ Physiol 290, H2472-2479.

36. Stein G H, Drullinger L F, Soulard A, Dulic V (1999) Differential Roles for Cyclin-Dependent Kinase Inhibitors p21 and p16 in the Mechanisms of Senescence and Differentiation in Human Fibroblasts. Mol Cell Biot 19, 2109-2117.

37. Stein S C, Woods A, Jones N A, Davison M D, Carling D (2000) The regulation of AMP-activated protein kinase by phosphorylation. Biochem J 345Pt3, 437-443.

38. Taussig R, Iniguez-Lluhi J A, Gilman A G (1993) Inhibition of adenylyl cyclase by Gi alpha. Science 261, 218-221.

39. Wang W, Yang X, Lopez de Silanes I, Carling. D, Gorospe M (2003) Increased AMP:ATP ratio and AMP-activated protein kinase activity during cellular senescence linked to reduced HuR function. J Biol Chem 278, 27016-27023.

40. Warden S M, Richardson C, O'Donnell J, Jr, Stapleton D, Kemp B E, Witters L A (2001) Post-translational modifications of the beta-1 subunit of AMP-activated protein kinase affect enzyme activity and cellular localization. Biochem J 354, 275-283.

41. Witters L A, Kemp B E (1992) Insulin activation of acetyl-CoA carboxylase accompanied by inhibition of the 5′-AMP-activated protein kinase. J Biol Chem 267, 2864-2867.

42. Woods A, Dickerson K, Heath R, Hong S P, Momcilovic M, Johnstone S R, Carlson M, Carling D (2005) Ca2+/calmodulin-dependent protein kinase kinase-β acts upstream of AMP-activated protein kinase in mammalian cells. Cell Metabolism 2, 21.

43. Woods A, Johnstone S R, Dickerson K, Leiper F C, Fryer L G, Neumann D, Schlattner U, Wallimann T, Carlson M, Carling D (2003a) LKB1 is the upstream kinase in the AMP-activated protein kinase cascade. Curr Biol 13, 2004-2008.

44. Woods A, Vertommen D, Neumann D, Turk R, Bayliss J, Schlattner U, Wallimann T, Carling D, Rider M H (2003b) Identification of Phosphorylation Sites in AMP-activated Protein Kinase (AMPK) for Upstream AMPK Kinases and Study of Their Roles by Site-directed Mutagenesis. J Biol Chem 278, 28434-28442.

45. Yeo E J, Hwang Y C, Kang C M, Choy H E, Park S C (2000) Reduction of UV-induced cell death in the human senescent fibroblasts. Mol Cells 10, 415-422.

46. Yeo E J, Jang I S, Lim H K, Ha K S, Park S C (2002) Agonist-specific differential changes of cellular signal transduction pathways in senescent human diploid fibroblasts. Exp Gerontol 37, 871-883.

1. A composition for regulating cellular senescence in senescent cells comprising lysophosphatidic acid and adenylyl cyclase inhibitor as active ingredients.

2. The composition according to claim 1, wherein the adenylyl cyclase inhibitor is selected from the group consisting of 2′,5′-dideoxyadenosine, cis-N-(2-phenylcyclopentyl)azacyclotridec-1-en-2-amine, and 9-(tetrahydro-2′-furyl)adenine.

3. The composition according to claim 1, wherein the effective dose of the lysophosphatidic acid is 1-50 μM.

4. The composition according to claim 1, wherein the effective dose of the adenylyl cyclase inhibitor is 1-500 μM.

5. The composition according to claim 1, wherein the senescent cell is derived from human cell.

6. A method for regulating cellular senescence containing the step of treating effective dose of lysophosphatidic acid and adenylyl cyclase inhibitor to senescent cells:

7. The method for regulating cellular senescence according to claim 6, wherein the adenylyl cyclase inhibitor is selected from the group consisting of 2′,5′-dideoxyadenosine, cis-N-(2-phenylcyclopentyl)azacyclotridec-1-en-2-amine, and 9-(tetrahydro-2′-furyl)adenine.

8. The method for regulating cellular senescence according to claim 6, wherein the effective dose of the lysophosphatidic acid is 1-50 μM.

9. The method for regulating cellular senescence according to claim 6, wherein the effective dose of the adenylyl cyclase inhibitor is 1-500 μM.

10. The method for regulating cellular senescence according to claim 6, wherein the senescent cell is derived from human cell.

11. A method for regulating cellular senescence of a subject in need of regulating cellular senescence containing the step of administering effective dose of lysophosphatidic acid and adenylyl cyclase inhibitor to the subject.
